(19) United States
(12) Patent Application Publication
(10) Pub. No.: US 2016/0228687 A1
(43) Pub. Date: Aug. 11, 2016

(54) NEEDLELESS CONNECTOR MODULE

(71) Applicant: LILY MEDICAL CORPORATION, MIAOLI COUNTY (TW)

(72) Inventors: YUNG-HUNG CHIH, NEW TAIPEI CITY (TW); HSIEN-CHIH TSAI, TAIPEI CITY (TW); CHIH-WEN LI, TAOYUAN CITY (TW); FENG-YANG HSIEH, TAICHUNG CITY (TW)

(21) Appl. No.: 14/829,721

(22) Filed: Aug. 19, 2015

(30) Foreign Application Priority Data
Feb. 9, 2015 (TW) 104104238

(51) Int. Cl. A61M 39/10; A61M 39/22

(52) U.S. Cl. CPC A61M 39/10; A61M 39/22; A61M 2039/1066

(57) ABSTRACT

A needleless connector module, including: a flow guiding member having a flow guiding channel connecting to a upper portion of the flow guiding member, the upper portion including an ultrasonic welding portion thereon; an elastic valve; and a sleeve tube having an inner annular bottom surface formed on an inner wall of a base portion of the sleeve tube, an limiting portion of annular wall being defined by the inner wall of the base portion, the sleeve tube is sleeved on the elastic valve and assembled with the flow guiding member to allow the inner annular bottom surface to contact with the ultrasonic welding portion and let the limiting portion of the annular wall annularly cover a base side portion of the flow guiding member, so as to improve success of ultrasonic welding between the ultrasonic welding portion and the limiting portion of annular wall.

### BACKGROUND OF THE INVENTION

1. Field of the Invention

The present disclosure relates to a needleless connector module; in particular, to a needleless connector module associated with the technical field of injector devices.

2. Description of Related Art

Needleless connector modules are produced mainly by ultrasonic welding so that components in the module can be connected with each other. However, the components are generally so small and some partial locations of the components are so fragile that abnormal transformation during manufacturing and defective fracture can both result.

The more quantity of the components, the more difficulty associated with welding connections by ultrasound. In particular, before the components undergo ultrasonic welding, a pre-assembling procedure among the components needs to be carried out to position the positions going to be welded among the components precisely and the ultrasonic welding can then performed. However, the components are so small as to make the pre-assembling more difficult. How to maintain the pre-assembling precision of the component moving along the processing line to achieve a good welding result is a tough issue that needs to be overcome. Especially when the needleless connector module includes too many components, not only does the number of times as well as the number of processes needed for ultrasonic welding becomes more and more complicated, but also the stability of pre-assembling is massively lowered leading to difficulty of ultrasonic welding and of improving good yield rate. During the ultrasonic welding procedure, heat could be generated to damage each of the components of the module. However, the more quantity of the components, the more frequency with which the ultrasonic welding needs to be performed. Thus, damages to each of the components by the ultrasonic welding can easily result during the process of the manufacture. More costs of molding will result due to the increase of the components needed. Hence, how to decrease the quantity of the needed components of the module to achieve the best pre-assembling effect, to decrease the frequency of the welding, reduce the difficulty of the welding, and raise the good yield rate and minimize the unnecessary cost, so as to enforce the competitive power of the product, is a big issue long to be improved.

Hence, the present inventors believe the above mentioned disadvantages can be overcome, and through devoted research combined with application of theory, finally proposes the present disclosure which has a reasonable design and effectively improves upon the above mentioned disadvantages.

### SUMMARY OF THE INVENTION

The object of the present disclosure is to provide a needleless connector module capable of improving the problem of too many components within the module as well as the difficulties of pre-assembling and ultrasonic welding.

In order to achieve the aforementioned objects, the present disclosure provides a needleless connector module comprising: a flow guiding member having a flow guiding channel formed along a central axis of the flow guiding member, the flow guiding channel connecting to a upper portion and a lower portion of the flow guiding member, a flow guiding tube being disposed on the upper portion and connected to the flow guiding channel, an edge of the upper portion including an ultrasonic welding portion thereon; an elastic valve being disposed on the upper portion and sleeved on the flow guiding tube, a slit being formed on a top surface of the elastic valve, a receiving orifice being hidden inside of the slit of the elastic valve; and a sleeve tube having an inner annular bottom surface formed on an inner wall of a base portion of the sleeve tube, the inner annular bottom surface centripetally extending relative to the base portion to define a base inner bore, a base outer bore being defined by the base portion of the sleeve tube, the base outer bore being larger than the base inner bore, a limiting portion of annular wall being defined by the inner wall of the base portion, wherein the sleeve tube is sleeved on the elastic valve and assembled with the flow guiding member to allow the inner annular bottom surface to be able to contact with the ultrasonic welding portion and let the limiting portion of annular wall annularly cover a base side portion of the flow guiding member, so as to allow the sleeve tube to be stably disposed on the flow guiding member and improve success of ultrasonic welding between the ultrasonic welding portion and the limiting portion of the annular wall; wherein a main chamber is defined as the sleeve tube is assembled with the flow guiding member and the elastic valve is in the main chamber.

Through the abovementioned technical features, the present disclosure can achieve a good pre-assembling effect by means of using less components, so as to lower down the difficulty of ultrasonic welding, raise the good yield rate of the product and minimize unnecessary costs.

In order to further the understanding regarding the present disclosure, the following embodiments are provided along with illustrations to facilitate the disclosure of the present disclosure.

### BRIEF DESCRIPTION OF THE DRAWINGS

FIG. 1A shows a perspective view from above of the needleless connector module according to the first embodiment of the instant disclosure;

FIG. 1B shows a perspective view from below of the needleless connector module according to the first embodiment of the present disclosure;

FIG. 2A shows an exploded view from above of the needleless connector module according to the first embodiment of the instant disclosure;

FIG. 2B shows an exploded view from below of the needleless connector module according to the first embodiment of the instant disclosure;

FIG. 3A shows a cross sectional view of the needleless connector module before being operated according to the first embodiment of the instant disclosure;

FIG. 3B shows a cross sectional view of the needleless connector module after being operated according to the first embodiment of the instant disclosure;

FIG. 3C shows a cross sectional view of the grasping inner wall of the needless connector module according to another embodiment of the instant disclosure;

FIG. 4A shows a perspective view from above of the needleless connector module according to the second embodiment of the instant disclosure;

FIG. 4B shows a perspective view from below of the needleless connector module according to the second embodiment of the instant disclosure;

FIG. 4C shows a partially exploded view from above of the needleless connector module according to a preferred example of the second embodiment of the instant disclosure;

FIG. 4D shows a partially exploded view from below of the needleless connector module according to a preferred example of the second embodiment of the instant disclosure;

FIG. 5A shows a perspective view from above of the needleless connector module according to another preferred example of the second embodiment of the instant disclosure;

FIG. 5B shows a perspective view from below of the needleless connector module according to another preferred example of the second embodiment of the instant disclosure;

FIG. 5C shows a partially exploded view from above of the needleless connector module according to another preferred example of the second embodiment of the instant disclosure;

FIG. 5D shows a partially exploded view from below of the needleless connector module according to another preferred example of the second embodiment of the instant disclosure;

FIG. 6A shows a perspective view from above of the needleless connector module according to another preferred example of the second embodiment of the instant disclosure;

FIG. 6B shows a perspective view from below of the needleless connector module according to another preferred example of the second embodiment of the instant disclosure;

FIG. 6C shows a partially exploded view from above of the needleless connector module according to another preferred example of the second embodiment of the instant disclosure; and

FIG. 6D shows a partially exploded view from below of the needleless connector module according to another preferred example of the second embodiment of the instant disclosure.

### DETAILED DESCRIPTION OF THE PREFERRED EMBODIMENTS

### First Embodiment

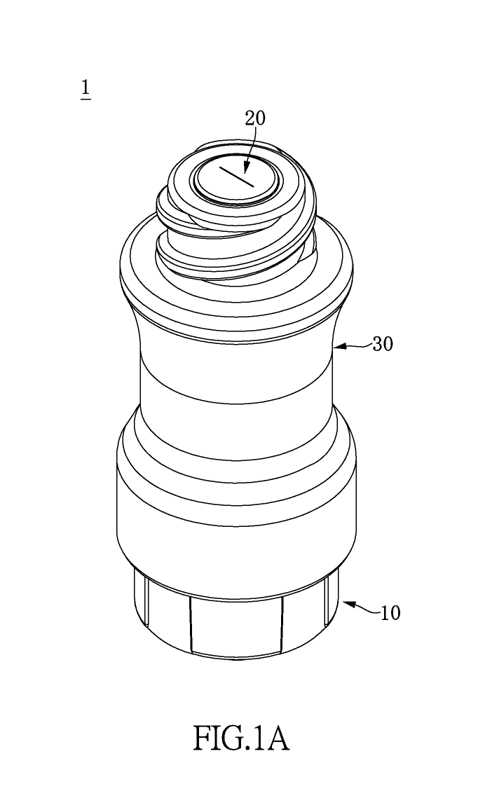

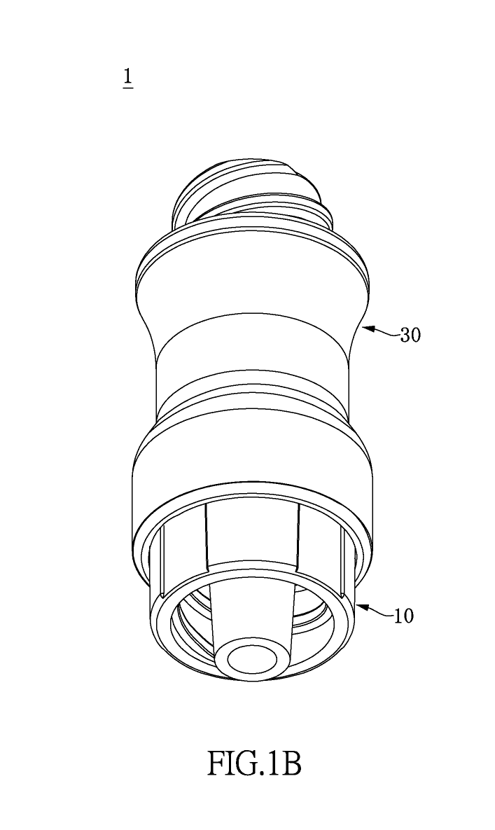

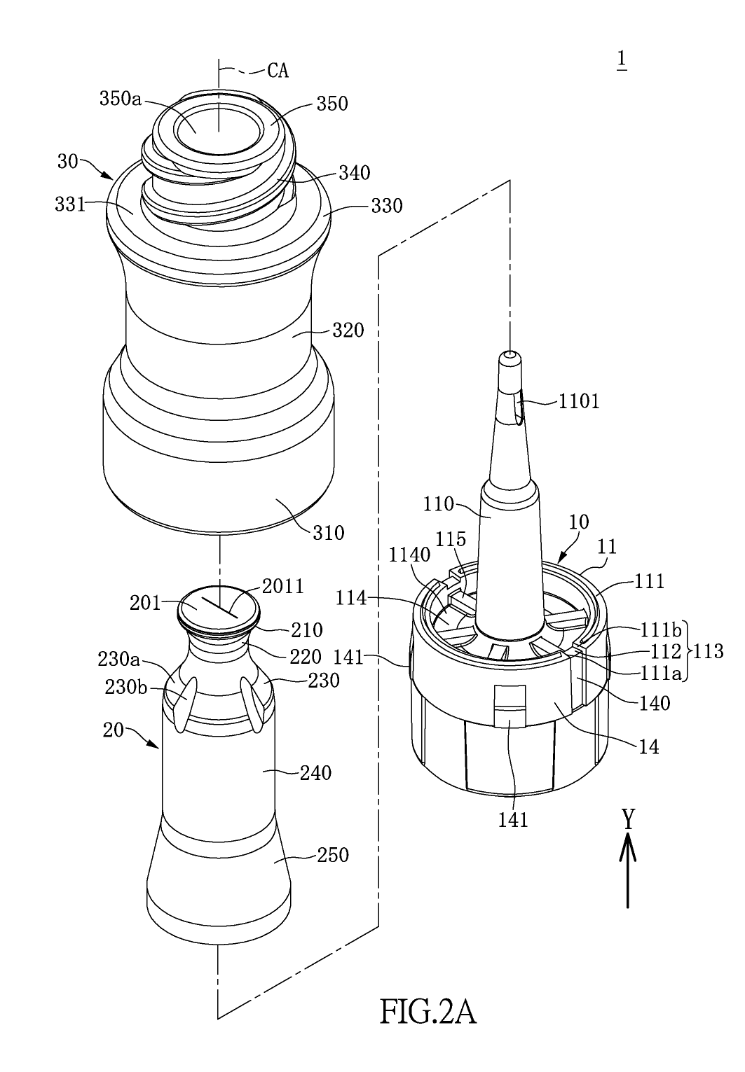

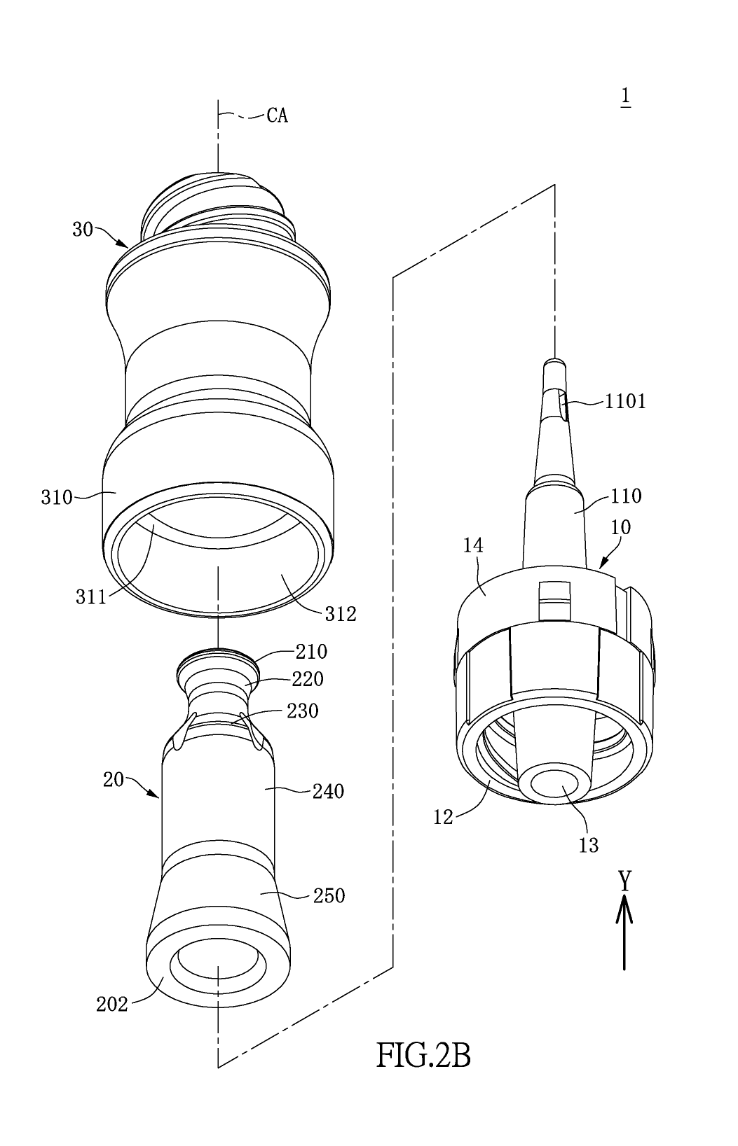

Please refer to FIGS. 1A, 1B, 2A and 2B, the present disclosure provides a needleless connector module 1 of three pieces including a flow guiding member 10, an elastic valve 20, and a sleeve tube 30.

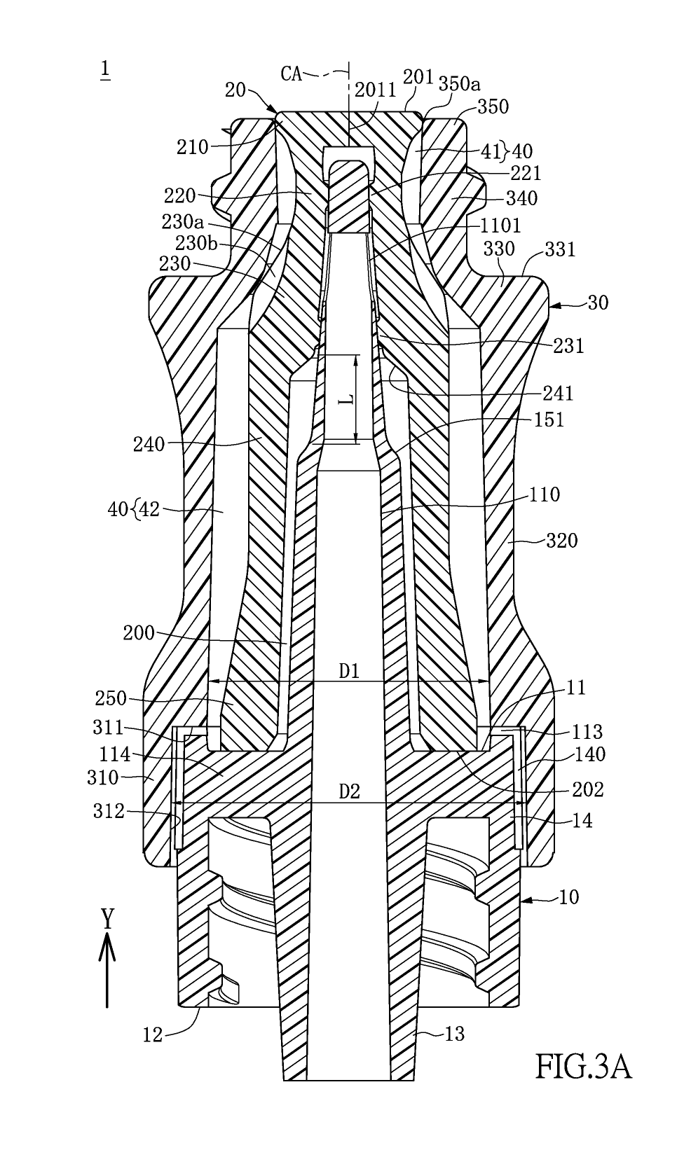

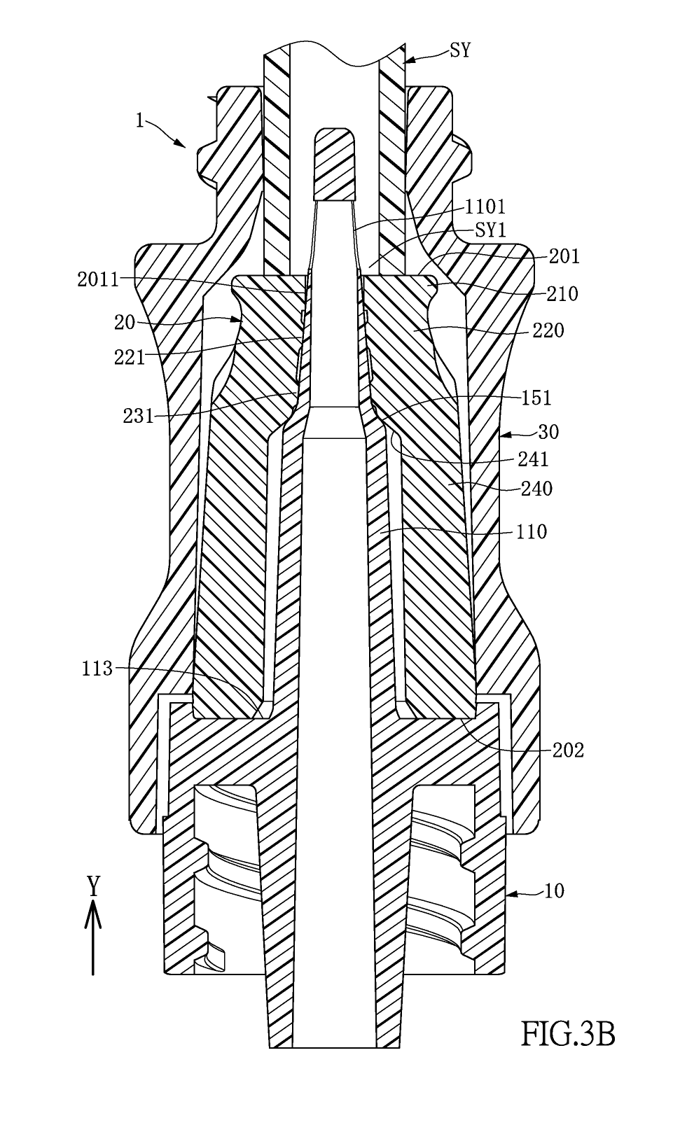

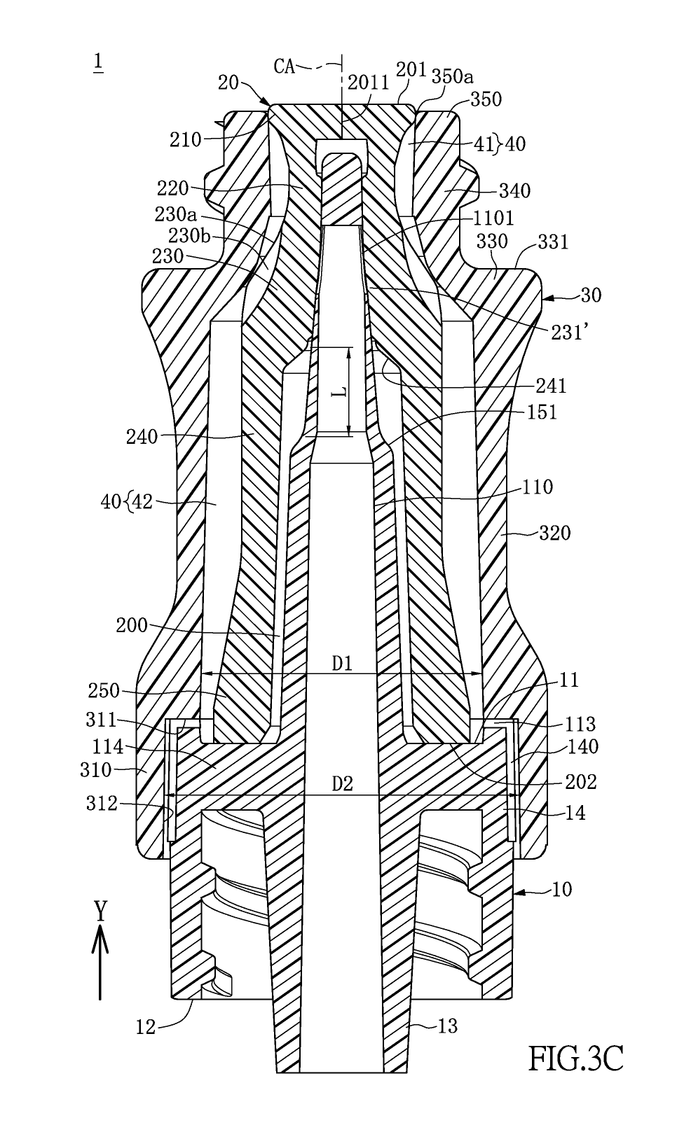

Please refer to FIGS. 2A, 2B and 3A. A flow guiding channel 13 is formed along a longitudinal axis Y of the flow guiding member 10. The flow guiding channel 13 connects to an upper portion 11 and a lower portion 12 of the flow guiding member 10, or preferably, the flow guiding channel 13 is formed along a central axis CA of the flow guiding member 10. Basically, the direction of the central axis CA is parallel to the longitudinal axis Y. The flow guiding channel 13 connects with an upper portion 11 and a lower portion 12 of the flow guiding member 10. A flow guiding tube 110 is disposed on the upper portion 11. In other words, the flow guiding tube 110 is protruded from the upper portion 11 and connected to the flow guiding channel 13. An edge of the upper portion 11 includes an ultrasonic welding portion 111 formed thereon.

As shown on FIGS. 2A, 2B and 3A, the elastic valve 20 is disposed on the upper portion 11 and sleeves on the flow guiding tube 110. A top surface of the elastic valve 20 is opened to form a slit 2011. As the elastic valve 20 sleeves on the flow guiding tube 110, a receiving orifice 1101 is opened on two relative side walls on a free end (label not shown) upwardly from the flow guiding tube 110 and is connected with the flow guiding tube 110 downwardly. The receiving orifice 1101 of the flow guiding tube 110 can be hidden inside of the elastic valve 20 and under the slit 2011. Preferably, the receiving orifice 1101 also connects with the perforation of the two relative walls.

Please refer to FIGS. 2A, 2B and 3A. An upper location of an inner wall of a base portion 310 of the sleeve tube 30 is extended toward the central axis CA to form an inner annular bottom surface 311. The inner annular bottom surface 311 indents toward the central axis CA so that a base inner bore D1 is defined. A base outer bore D2 is also defined by the base portion 310 of the sleeve tube 30. The diameter of the base outer bore D2 is larger than that of the base inner bore D1. The inner wall of the base portion 310 also defines a limiting portion of annular wall 312. As the elastic valve 20 is disposed on the flow guiding member 10 to undergo an assembling procedure, the sleeve tube 30 can be sleeved outside of the elastic valve 20 and assembled with the flow guiding member 10 so that the inner annular bottom surface 311 contacts with the ultrasonic welding portion 111. The limiting portion of annular wall 312 can annularly cover the base side portion 14 of the flow guiding member 10, so as to allow the sleeve tube 30 to be disposed on the flow guiding member 10 more stably. Furthermore, when the instant disclosure is undergoing the procedure of production, the limiting portion of annular wall 312 of the base portion 310 as well as the inner annular bottom surface 311 above the limiting portion of annular wall 312 can be viewed as being equivalent to a cap-form joint structure (label not shown), so as to be used to cap on and cover a part of the base side portion 14 of the flow guiding member 10, helping the sleeve tube 30 to be located on the flow guiding member 10, having been assembled with the elastic valve 20 more stably to form a stable pre-assembling structure. Hence, before the ultrasonic welding, it is necessary to make the pre-assembling of the sleeve tube 30 and the flow guiding member 10 more stable to effectively prevent the relative shifting between the sleeve tube 30 and the flow guiding member 10 from occurring during the producing process or movement in the production line. Thus, meaningful contact between the inner annular bottom surface 311 and the ultrasonic welding portion 111 can be also maintained and improved, helping to raise the success rate of ultrasonic welding between the ultrasonic welding portion 111 and the inner annular bottom surface 311. When the sleeve tube 30 is assembled with the flow guiding member 10, a main chamber 40 is defined and the elastic valve 20 having been assembled with the flow guiding tube 110 is in the main chamber 40.

Please refer to FIG. 2A, the edge of the upper portion 11 also includes a non-welding portion 112. The non-welding portion 112 is formed on the ultrasonic welding portion 111 and intercepts the ultrasonic welding portion 111 so that the ultrasonic welding portion 111 forms two cross sections (111a, 111b) opposing to each other. The non-welding portion 112 is lower than the ultrasonic welding portion 111 so that a non-welding notch 113 is defined by the non-welding portion 112 and the two cross sections (111a, 111b) opposing to each other. The non-welding notch 113 is connected to the main chamber 40. As shown in FIG. 2A, if the upper portion 11 roughly defines a circle and the circle has a circle center (label not shown), the central axis CA basically passes through the circle center and another non-welding notch (label not shown), opposite to the location of the non-welding portion 113 on a diameter passing through the circle center and the non-welding portion 113, can be found. In the instant embodiment, there can be two non-welding portions 113, but it is not limited thereto. There can also be only one non-welding portion 113 or more than two non-welding portions 113. The non-welding portions 113 can be spacedly disposed along the ultrasonic welding portion 111, or the location of each of the non-welding portions 113 can be equally spaced along the ultrasonic welding portion 111, but of course, it is not limited thereto. Please refer to FIG. 3A. Shown in a diagram of a cross-section view, the non-welding notch 113 is able to connect to the main chamber 40.

Please refer to FIG. 2A. A side trench 140 is formed on the base side portion 14. As shown in FIG. 3A, when the assembling of the instant disclosure is completed so that the cap-form structure is formed by covering the outside of the base side portion 14 with the sleeve tube 30, the formation of the cap-form structure is contributed to by the combination of the inner annular bottom surface 311 and the limiting portion of the annular wall 312. The side trench 140 can downwardly connect to the outside of the needleless connector module 1 and upwardly connect to the non-welding notch 113. The main chamber 40 of the needleless connector module 1 can connect to the outside of the needleless connector module 1 through the non-welding notch 113 and the side trench 140. Once the instant disclosure is produced and is undergoing a sterilizing procedure for subsequent sale, the structure of the main chamber 40 capable of connecting to the outside is helpful for the entrance of high-temperature as well as high-pressure sterile vapor, so as to avoid the possible risks of incomplete sterilization when the needleless connector module 1 goes out from the factory.

Please refer to FIGS. 2A, 2B, and 3A. The elastic valve 20 is hollow and respectively defines a valve head portion 210, valve neck portion 220, valve shoulder portion 230, a valve waist portion 240 and a valve base portion 250 from the top surface 201 to the annular bottom surface 202 of the elastic valve 20. Please refer to FIGS. 2A and 3A. An insertion opening 350a is formed at a top terminal 350 of the sleeve tube 30. As the sleeve tube 30 is sleeved on the elastic valve 20, the valve head portion 210 is just located in the insertion opening 350a and blocks the insertion opening 350a. Please refer to FIGS. 2A and 3A. A tube head portion 340, tube shoulder portion 330 and tube main portion 320 are respectively defined from the insertion opening 350a to the tube base portion 310. Thus, it is known that the tube main portion 320 downwardly connects to the tube base portion 310. An upper interspace 41 is defined in the main chamber 40 by inner walls of the tube head portion 340 and the tube shoulder portion 330, and outer walls of the valve head portion 210 and valve neck portion 220. A lower interspace 42 is defined in the main chamber 40 by inner walls of the tube shoulder portion 330 and the tube waist/main portion 320, and outer walls of the valve shoulder portion 230, the valve waist portion 240 and the valve base portion 250. A part of the outer wall of the valve shoulder portion 230 is defined as a shoulder incline 230a and the shoulder incline 230a abuts the inner wall of the tube shoulder portion 330. Another part of the outer wall of the valve shoulder portion 230 is recessed to form an air duct recess 230b. The air duct recess 230b is more recessed than the shoulder incline 230a and connects with the upper interspace 41 and the lower interspace 42. Preferably, the quantity of the air duct recess 230b is not limited to be only one. The quantity of the shoulder incline 230a is not limited to be only one, either. Preferably, the air duct recess 230b and the shoulder incline 230a can be tandem-repetitively formed on the valve shoulder portion 230 around the central axis CA so that the quantities of the shoulder incline 230a and the air duct recess 230b are not limited thereto. In other words, an air duct recess 230b, a shoulder incline 230a adjacent to the air duct recess 230b, another air duct recess (label not shown) adjacent to the shoulder incline 230a and another shoulder incline (label not shown) adjacent to the another air duct recess can be repeatedly formed on the valve shoulder portion 230 around the central axis CA till a cycle of a spire is completed by such one by one arrangement. The air duct recess 230b helps to form the interconnection between the upper interspace 41 and the lower interspace 42 so that the air duct recess 230b helps the high-temperature and high-pressure steam for sterilization to get to the upper interspace 41 all the way through the side trench 140 and the lower interspace 42 without being hindered and to accomplish a complete sterilization. As a result, dead space unable to be sterilized inside the main chamber 40 can be avoided.

As shown in FIG. 3A, an upper surface of the tube shoulder portion 330 is defined as a shoulder upper surface 331. The shoulder upper surface 331 is further defined as a weldable portion for being treated with ultrasonic welding, so as to allow the annular bottom surface 311 to be welded with the weldable portion. When the pre-assembling of the flow guiding member 10, the elastic valve 20 and the sleeve tube 30 is completed, the advantage for manufacturing due to the stable pre-assembling structure results. Also, the ultrasound can efficiently reach the ultrasonic welding portion 111 (as shown in FIG. 2A) without causing bad influences on the stability of the pre-assembling structure of the instant disclosure so that the welding work between the inner annular bottom surface 311 and the ultrasonic welding portion 111 goes smoothly (as shown in FIGS. 2A, 2B, and 3A).

Please refer to FIG. 2A. Several bumps 141 are protruded in a slight lateral direction from the base side portion 10. The several bumps 141 are spacedly disposed on the base side portion 10 along a putative spire and/or circular path (not shown) on the flow guiding member 10. The several bumps 141 are able to form a tight fit contact with the limiting portion of the annular wall 312, which can also be viewed as the inner wall of the tube base portion 310. Preferably, the quantity of the several bumps 141 could be at least three. Take the three bumps 141 for example, as the three bumps are spacedly arranged on the flow guiding member 10 along the spire path, any two of the three bumps 141 adjacent to each other form a 120 degree included angle with a center of the circular path. In other words, from a view of a bird's eye (not shown), the at least three bumps 141 are symmetrically arranged according to a symmetry axis, e.g. the central axis CA. In the instant embodiment, the bumps 141 belong to a part of the base side portion 14 and are elements of the base side portion 14. Though the limiting portion of the annular wall 312 causes an engaging effect by the contact with the bumps 141, from another viewpoint, even though the bumps 141 exist, it is equivalent in meaning to say the limiting portion of the annular wall 312 covers the base side portion 14.

Please refer to FIGS. 3A and 3B. A part of the inner wall of the valve waist portion 240 is extended and expanded along a direction toward the annular bottom surface 202. This is to say that the valve waist portion 240 downwardly extends and laterally expands its wideness to form a slant expansion inner wall 241. The outer wall of the flow guiding tube 110 includes a slant expansion slope 151. The slant expansion slope 151 slantingly expands toward a direction of the upper portion 11 so that the slope (or called gradient of slope) of some parts of the slant expansion slope 151 belonging to the flow guiding tube 110 is smaller than the slope of the outer wall defined from some other parts of the flow guiding tube 110 from top to down of the flow guiding tube 110 and the gradient slope of the slant expansion slope 151 is larger than zero. As shown in FIG. 3A, under a first usage condition, the receiving orifice 1101 is received in the valve neck portion 220 and the slant expansion slope 151 is under the slant expansion inner wall 241 with a predetermined distance L. As shown in FIG. 3B, under a second usage condition, the top surface 201 is pressed by a syringe SY so that the valve head portion 210 is compressed downwardly and the elastic valve 20 is then compressed to bring the slant expansion inner wall 241 to move for the aforementioned predetermined distance to fittingly contact with the slant expansion slope 151, allowing the receiving orifice 1101 to be exposed out of the slit 2011 and to be connected with an injection opening SY1 of the syringe SY.

Please refer to FIG. 3A. A grasping inner wall 231 is formed on the slant expansion inner wall 241 to an inner wall of the valve shoulder portion 230 and is protruded toward the flow guiding tube 110. As the elastic valve 20 is assembled to the flow guiding tube 1, the grasping inner wall 231 is under the receiving orifice 1101. As the first usage condition shown in FIG. 3A, the grasping inner wall 231 is under the receiving orifice 1101. As the second usage condition shown in FIG. 3B, the grasping inner wall 231 can be pushed downwardly to press the slant expansion slope 151 with an action force, and as it is turned back to the first usage condition from the second usage condition, in other words, as the syringe SY is drawn out from the insertion opening 350a, a reaction force relative to the action force resulting from the slant expansion slope 151 is applied to the grasping inner wall 231, so as to decompress the elastic valve 20 and make the elastic valve 20 recover to the condition shown in FIG. 3A. In addition, another grasping inner wall 221 is protruded toward the flow guiding tube 110 from the inner wall of the valve neck portion 220. This grasping inner wall 221 is located above the receiving orifice 1101. It is different from the grasping inner wall 231 only due to the positions they are respectively located at, however the respective functions are similar to each other. Please refer to FIG. 3C, another grasping inner wall 221 integrates with the grasping inner wall 231 and to form a grasping inner wall 231′. In other words, the grasping inner wall 231′ projects towards flow guiding tube 110 and is formed between the slant expansion inner wall 241 and the inner wall of the valve neck portion 220.

Please refer to FIG. 2A. The upper portion 11 includes an upper platform 114 and several ribs 115 protruded from the upper platform 114. The several ribs 115 are arranged in a centric direction based on the central axis CA, and the terminals away from the central axis respectively on the several ribs 115 are spacedly separated along a circle. The annular bottom surface 202 of the elastic valve 20 is located on the several ribs 115, rendering the main chamber 40 to connect with inside of the elastic valve 20. Spacers 1140 are respectively defined between any two of the several ribs 115 among the several ribs 115. It is revealed from FIG. 2A that as the elastic valve 20 is put on the flow guiding member 10 to sleeve on the flow guiding tube 110 and the sleeve tube 30 is assembled to the flow guiding member, the spacers 1140 shown in FIG. 2A are able to connect with the valve inner chamber 200 of the elastic valve 20 and the lower interspace 42 of the main chamber 40. Once the elastic valve 20 is elevated by the ribs 115, the vapor of high-temperature and high-pressure for sterilizing is assisted to enter the valve inner chamber 200 to avoid the risks of incomplete sterilization.

### Second Embodiment

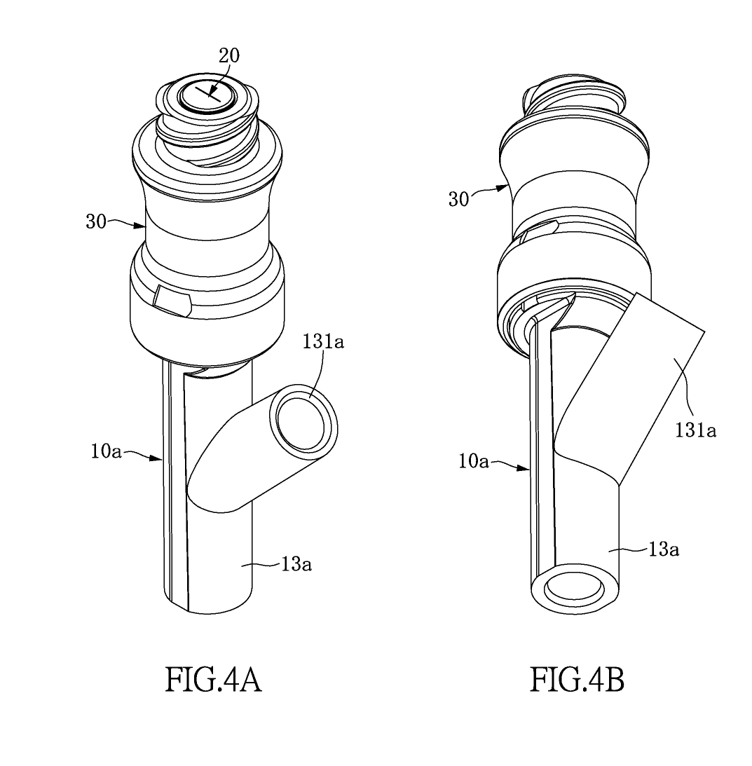

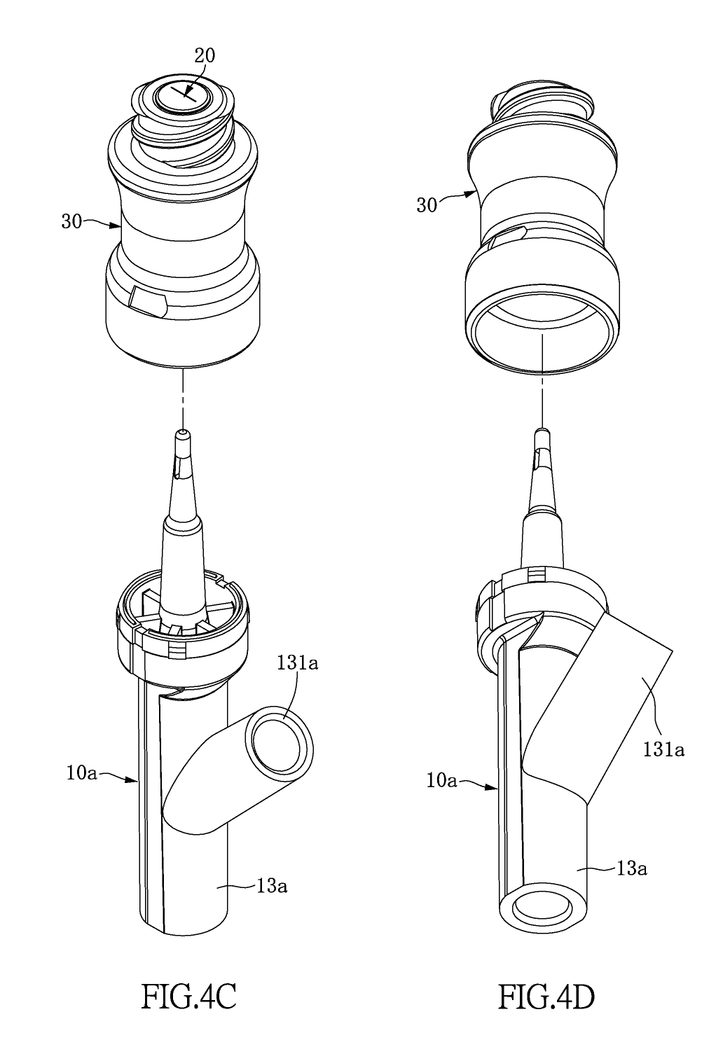

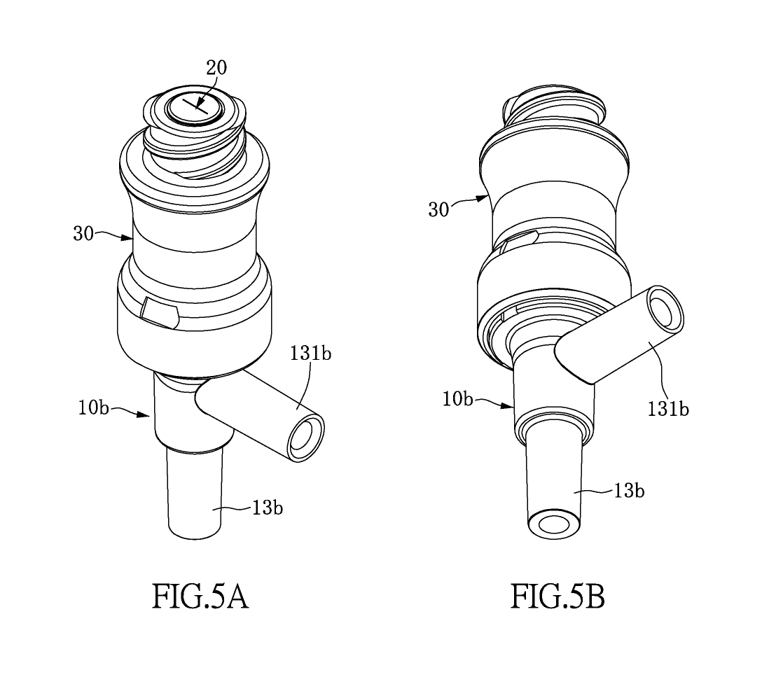

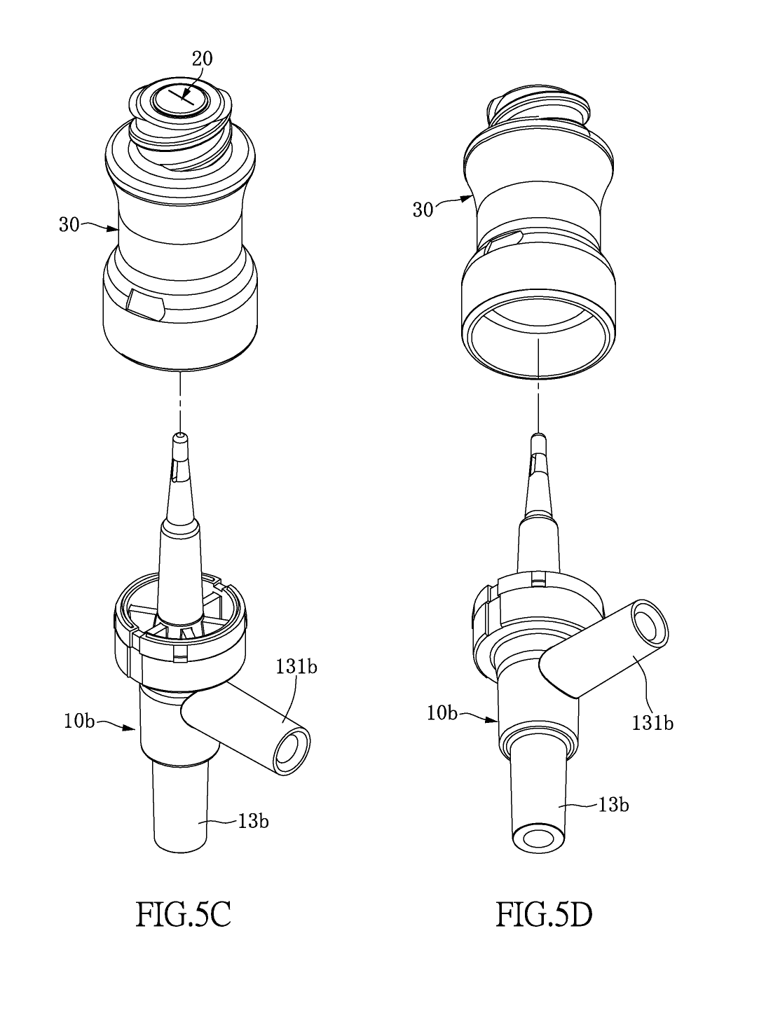

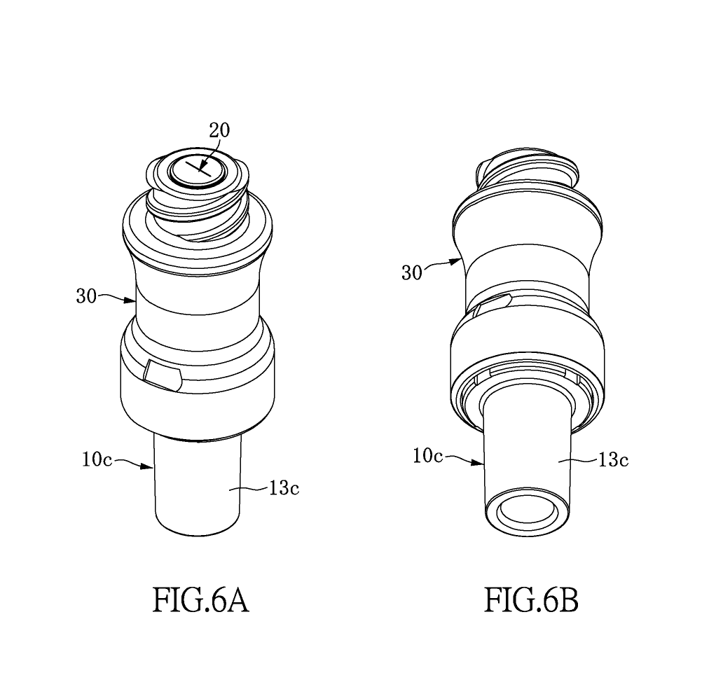

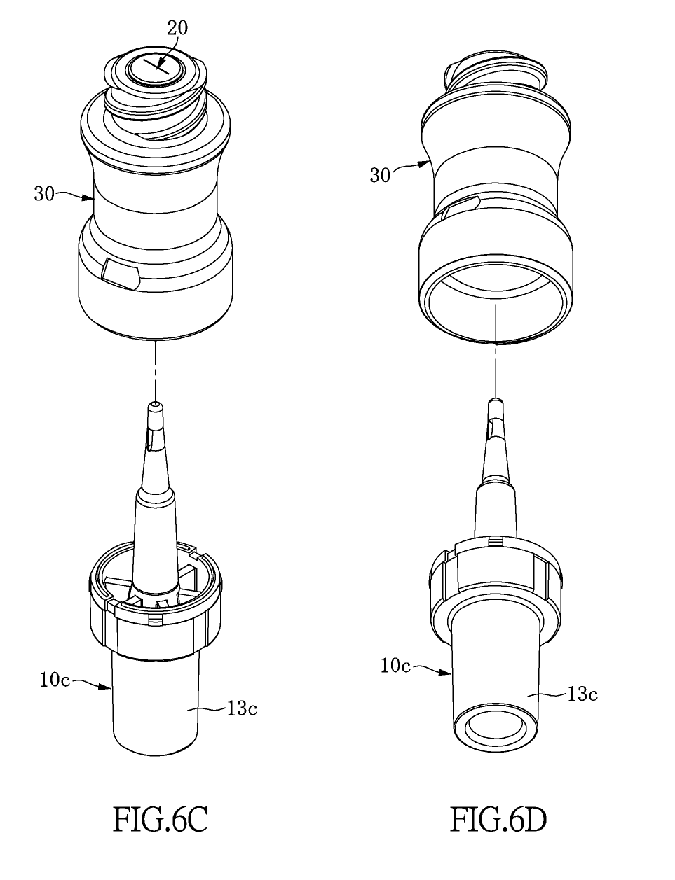

Please refer to FIGS. 4A, 4B, 4C and 4D. In a preferred embodiment, the flow guiding channel 13a of the flow guiding member 10a is the only element different from that of the aforementioned embodiment and other elements in this embodiment are basically the same to those of the aforementioned embodiments. The flow guiding channel 13a of the instant embodiment is connected with a branch channel 131a. The branch channel 131a and the flow guiding channel 13a together form a Y shaped channel, or in other words, a Y shaped pipe. The flow guiding channel 13a can be a plug-type pipe. In addition, in another preferred embodiment shown in FIGS. 5A, 5B, 5C and 5D, the flow guiding member 10b is the only element different from the aforementioned embodiments and other elements are the same to those of the aforementioned embodiments. The branch channel 131b can be perpendicular to the flow guiding channel 13b of the flow guiding member 10b. Furthermore, in another preferred embodiment as shown in FIGS. 6A, 6B, 6C, and 6D, the flow guiding member 10c is the only element different to that of the aforementioned embodiment and other elements are basically the same to those of the aforementioned embodiments. The flow guiding channel 13c of the flow guiding member 10 can be a simple plug-type pipe without any other branched channel and is different to the one disclosed in the first embodiment which has a bolted structure formed thereon. As shown in FIGS. 1B, 4B, 5B and 5C, the aforementioned flow guiding channels (13, 13a, 13b, 13c) can be used for connecting with other downstream pipes (not shown), and such downstream pipes are mainly for in vivo connection for transferring liquid or medicine in liquid form to patients. The use of the branch channels (131a or 131b) depends on actual requirements. Thus, medical liquid or needed liquid, such as nutrition supplement or glucose solution etc., can be delivered through the branch channels (131a or 131b).

In sum, the needleless connector module of the instant disclosure overall includes three main components. Unnecessary components can be decreased, so as to simplify needed elements, reduce costs, and simplify assembling as well as welding work.

Due to the simplification and optimization of the needed components, the structure of pre-assembling of the instant disclosure can be more stable, raising the success of welding work and improving the yield quality.

The unique structure of the instant disclosure helps the entrance of high-temperature and high-pressure sterile vapor into the inside of the instant disclosure be easier. Blind spots unable to be sterilized are avoided and the public health as well as the medical security can be improved.

The descriptions illustrated supra set forth simply the preferred embodiments of the present disclosure; however, the characteristics of the present disclosure are by no means restricted thereto. All changes, alternations, or modifications conveniently considered by those skilled in the art are deemed to be encompassed within the scope of the present disclosure delineated by the following claims.

What is claimed is:

1. A needleless connector module comprising:
a flow guiding member having a flow guiding channel formed along a central axis of the flow guiding member, the flow guiding channel connecting to a upper portion and a lower portion of the flow guiding member, a flow guiding tube being disposed on the upper portion and connected to the flow guiding channel, an edge of the upper portion including an ultrasonic welding portion thereon;
an elastic valve being disposed on the upper portion and sleeved on the flow guiding tube, a slit being formed on a top surface of the elastic valve, a receiving orifice being hidden inside of the slit of the elastic valve; and
a sleeve tube having an inner annular bottom surface formed on an inner wall of a base portion of the sleeve tube, the inner annular bottom surface centripetally extending relative to the base portion to define a base inner bore, a base outer bore being defined by the base portion of the sleeve tube, the base outer bore being larger than the base inner bore, an limiting portion of annular wall being defined by the inner wall of the base portion,
wherein the sleeve tube is sleeved on the elastic valve and assembled with the flow guiding member to allow the inner annular bottom surface to be able to contact with the ultrasonic welding portion and let the limiting portion of annular wall annularly cover a base side portion of the flow guiding member, so as to allow the sleeve tube to be stably disposed on the flow guiding member and improve success of ultrasonic welding between the ultrasonic welding portion and the limiting portion of annular wall;
wherein a main chamber is defined as the sleeve tube is assembled with the flow guiding member and the elastic valve is in the main chamber.

2. The needleless connector module according to claim 1, wherein the edge of the upper portion comprises a non-welding portion formed thereon and intercepting the ultrasonic welding portion so that the ultrasonic welding portion forms two cross sections opposing to each other; the non-welding portion is lower than the ultrasonic welding portion so that a non-welding notch is defined by the non-welding portion and the two cross sections opposing to each other; and the non-welding notch is connected to the main chamber.

3. The needleless connector module according to claim 2, wherein a side trench is formed on the base side portion, one terminal of the side trench connecting to outside of the needleless connector module and the other terminal of the side trench connecting to the non-welding notch.

4. The needleless connector module according to claim 1, wherein the elastic valve is hollow and respectively defines a valve head portion, valve neck portion, valve shoulder portion, a valve waist portion and a valve base portion from the top surface to an annular bottom surface of the elastic valve; wherein an insertion opening is formed at a top terminal of the sleeve tube, the valve head portion being located in the insertion opening and blocks the insertion opening, wherein a tube head portion, tube shoulder portion and tube base portion, wherein an upper interspace is defined by an inner wall of the tube head portion and the tube shoulder portion and an outer wall of the valve head portion and valve neck portion, and an inner wall of the tube shoulder portion and the tube waist portion, and a lower interspace is defined by an inner wall of the tube shoulder portion and the tube waist portion and an outer wall of the valve waist portion and the valve base portion, a part of the outer wall of the valve shoulder portion being defined as a shoulder incline and the shoulder incline abutting the inner wall of the tube shoulder portion, another part of the outer wall of the valve shoulder portion being recessed to form an air duct recess, the air duct recess being more recessed than the shoulder incline and connecting the upper interspace and the lower interspace.

5. The needleless connector module according to claim 1, wherein several bumps are protruded from the base side portion and spacedly disposed on the base side portion, the several bumps forming a tight fit contact with the limiting portion of annular wall.

6. The needleless connector module according to claim 1, wherein the elastic valve is hollow and respectively defines a valve head portion, valve neck portion, valve shoulder portion, a valve waist portion and a valve base portion from the top surface to an annular bottom surface of the elastic valve, a part of an inner wall of the valve waist portion being extended and expanded along a direction toward the annular bottom surface to form a slant expansion inner wall; wherein an outer wall of the flow guiding tube includes a slant expansion slope slantingly expanded to a direction toward the upper portion,
wherein under a first usage condition, the receiving orifice is received in the valve neck portion and the slant expansion slope is under the slant expansion inner wall;
wherein under a second usage condition, the top surface is pressed by a syringe so that the valve head portion is compressed downwardly and the elastic valve is then compressed to bring the slant expansion inner wall to fittingly contact with the slant expansion slope, allowing the receiving orifice to be exposed out of the slit and to be connected with an injection opening of the syringe.

7. The needleless connector module according to claim 6, wherein a grasping inner wall is formed from the slant expansion inner wall to an inner wall of the valve shoulder portion and is protruded to the flow guiding tube, wherein under the second usage condition, the grasping inner wall is pushed downwardly to press the slant expansion slope with an action force, and as the second usage condition is turned to the first usage condition, a reaction force relative to the action force resulted from the slant expansion slope is applied to the grasping inner wall, so as to decompress the elastic valve.

8. The needleless connector module according to claim 4, wherein an upper surface of the tube shoulder portion is defined as a shoulder upper surface, the shoulder upper surface being defined as a weldable portion for being treated with ultrasonic welding, so as to allow the annular bottom surface to be welded with the weldable portion.

9. The needleless connector module according to claim 1, wherein the upper portion includes an upper platform and several ribs protruded from the upper platform, and the annular bottom surface is located on the several ribs, rendering the main chamber to connect with inside of the elastic valve.

10. The needleless connector module according to claim 1, wherein the flow guiding channel is connected with a branch channel, wherein the branch channel and the flow guiding channel together form a Y shaped channel or the branch channel is perpendicular to the flow guiding channel.
